(19) United States
(12) Patent Application Publication
(10) Pub. No.: US 2025/0131569 A1
(43) Pub. Date: Apr. 24, 2025

(54) SYSTEMS AND METHODS FOR DETECTING AND PREDICTING PHENOTYPIC MEASUREMENTS AND PHENOTYPES

(71) Applicant: THE TEXAS A&M UNIVERSITY SYSTEM, College Station, TX (US)

(72) Inventors: Seth C. MURRAY, Caldwell, TX (US); Alper ADAK, College Station, TX (US); Aaron James DESALVIO, Bryan, TX (US)

(21) Appl. No.: 18/921,809

(22) Filed: Oct. 21, 2024

Related U.S. Application Data
(60) Provisional application No. 63/591,572, filed on Oct. 19, 2023.

(51) Int. Cl. G06T 7/00; A01G 7/06; G01N 21/84; G01N 33/00; G06V 20/10; G06V 20/17

(52) U.S. Cl. CPC G06T 7/0016; A01G 7/06; G01N 21/84; G01N 33/0098; G06V 20/17; G06V 20/188; G01N 2021/8466; G06T 2207/10032; G06T 2207/20081; G06T 2207/30188

(57) ABSTRACT

An example system for predicting a phenotypic measurement includes a remote sensing platform comprising a remote sensor configured to capture image data, where the image data comprises a plurality of temporally-spaced images of a plant; and a computing device operably coupled to the remote sensing platform, where the computing device includes at least one processor and memory, the memory having computer-executable instructions stored thereon that, when executed by the at least one processor, cause the at least one processor to: input the image data into a prediction model; and determine, using the prediction model, a phenotypic measurement.

### CROSS REFERENCE TO RELATED APPLICATIONS

This application claims priority to, and the benefit of, U.S. Provisional Patent Application No. 63/591,572, filed Oct. 19, 2023, which is incorporated by reference herein in its entirety.

### STATEMENT REGARDING FEDERALLY FUNDED RESEARCH

This invention was made with government support under 2020-068013-32371 awarded by the National Institute of Food and Agriculture, 2021-67013-33915 awarded by the National Institute of Food and Agriculture, and USDA NIFA Hatch TEX07758 awarded by the National Institute of Food and Agriculture. The government has certain rights in the invention.

### BACKGROUND

Plant senescence (i.e., aging) refers to the process of plants aging, involving dying in annual plants and going dormant in perennials and trees. It can be important to detect and quantify the plant senescence phenotype, (which can include detecting which plants are in the period of time where a plant dries down and dies), and accurately predict plant senescence before it occurs. For example, in grains and annual plants, the end of plant senescence marks the end of the grain filling period, where the grain filling period correlates to the yield of the plant. As another example, stay green is a phenotype where plants have delayed plant senescence, and thus “stay green” longer, and can have increased yields. Improvements to the detection of different plant phenotypes like plant senescence and stay green can improve the prediction of crop yields, management practices, selecting better varieties and other valuable information. Moreover, improvements to the detection of different phenotypes can be used to better manage agricultural resources. A major challenge is objectively characterizing plant senescence and stay green and their progression over time.

### SUMMARY

In some aspects, the techniques described herein relate to a method of phenotypic measurement including: receiving image data from a remote sensing platform, wherein the image data includes a plurality of temporally-spaced images of a plant; inputting the image data into a prediction model; and determining, using the prediction model, a phenotypic measurement.

In some aspects, the techniques described herein relate to a method, wherein the prediction model includes a phenomic biomarker prediction model.

In some aspects, the techniques described herein relate to a method, wherein the prediction model includes a genomic, metabolomic, or proteomic prediction model.

In some aspects, the techniques described herein relate to a method, wherein the prediction model is a trained machine learning model.

In some aspects, the techniques described herein relate to a method, wherein the phenotypic measurement of interest includes plant senescence or stay green.

In some aspects, the techniques described herein relate to a method, further including determining a senescence score based on the plurality of image data.

In some aspects, the techniques described herein relate to a method, further including determining a dependent phenotypic trait of interest, wherein the dependent phenotypic trait of interest includes grain filling period.

In some aspects, the techniques described herein relate to a method further including modeling a trajectory of the phenotypic measurement using a mathematical or statistical model over time.

In some aspects, the techniques described herein relate to a method, wherein image data is collected at an altitude of less than or equal to 100 meters.

In some aspects, the techniques described herein relate to a method, wherein the image data includes images with a higher resolution than or equal to 25 cm per pixel.

In some aspects, the techniques described herein relate to a method, wherein the remote sensing platform includes an unoccupied aerial system (UAS).

In some aspects, the techniques described herein relate to a method, wherein the remote sensing platform includes a plurality of UASs.

In some aspects, the techniques described herein relate to a method, further including controlling the remote sensing platform based on the phenotypic measurement.

In some aspects, the techniques described herein relate to a method, further including adjusting the inputs to the plant based on the phenotypic measurement.

In some aspects, the techniques described herein relate to a method, further including selecting varieties based on phenomic prediction of senescence and stay green.

In some aspects, the techniques described herein relate to a method, wherein the remote sensing platform includes satellite, ground vehicles, robots, handheld sensors and related methods.

In some aspects, the techniques described herein relate to a system including: a remote sensing platform including a remote sensor configured to capture image data, wherein the image data includes a plurality of temporally-spaced images of a plant; and a computing device operably coupled to the remote sensing platform, wherein the computing device includes at least one processor and memory, the memory having computer-executable instructions stored thereon that, when executed by the at least one processor, cause the at least one processor to: input the image data into a prediction model; and determine, using the prediction model, a phenotypic measurement.

In some aspects, the techniques described herein relate to a system, wherein the prediction model includes a phenomic biomarker prediction model.

In some aspects, the techniques described herein relate to a system or claim 18, wherein the prediction model includes a genomic, metabolomic, or proteomic prediction model.

In some aspects, the techniques described herein relate to a system, wherein the prediction model is a trained machine learning model.

In some aspects, the techniques described herein relate to a system any one, wherein the phenotypic measurement includes plant senescence.

In some aspects, the techniques described herein relate to a system, further including determining a dependent phenotypic trait of interest, wherein the dependent phenotypic trait of interest includes grain filling period.

In some aspects, the techniques described herein relate to a system, wherein image data is collected at an altitude of less than or equal to 25 meters.

In some aspects, the techniques described herein relate to a system, wherein the image data includes images with a higher resolution than or equal to 25 cm per pixel.

In some aspects, the techniques described herein relate to a system, wherein the remote sensing platform includes an unoccupied aerial system (UAS).

In some aspects, the techniques described herein relate to a system, wherein the remote sensing platform includes a plurality of UASs.

In some aspects, the techniques described herein relate to a system, wherein the memory has further computer-executable instructions stored thereon that, when executed by the at least one processor, cause the at least one processor to: control the remote sensing platform based on the phenotypic measurement.

In some aspects, the techniques described herein relate to a system, wherein the memory has further computer-executable instructions stored thereon that, when executed by the at least one processor, cause the at least one processor to: adjust the inputs to the plant based on the phenotypic measurement.

In some aspects, the techniques described herein relate to a system, wherein the memory has further computer-executable instructions stored thereon that, when executed by the at least one processor, cause the at least one processor to: determine a senescence score or grain filling period based on the plurality of image data.

In some aspects, the techniques described herein relate to a system, wherein the remote sensing platform includes satellite, ground vehicles, robots, handheld sensors and related methods.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates an example method of predicting a phenotypic measurement, according to implementations of the present disclosure.

FIG. 2 illustrates an example system for predicting phenotypic measurements using remote sensing, according to implementations of the present disclosure.

FIG. 3 illustrates an example computing device.

FIG. 4 illustrates a series of unoccupied aerial system (UAS) flights that were performed using an example implementation of the present disclosure including observation of green and senesced plots.

FIG. 5 illustrates an example analysis of days to senescence and grain filling period in maize, according to an example implementation of the present disclosure.

FIG. 6 illustrates a table of example prediction models and notations, according to implementations of the present disclosure.

FIG. 7A illustrates an example variance component result for temporal senescence progression.

FIG. 7B illustrates an example variance component result for flowering times, plant heights, grain filling period, and days to senescence.

FIG. 7C illustrates example genotypic values by recombinant-inbred-line-population for temporal senescence.

FIG. 7D illustrates example genotypic values by each recombinant-inbred-line-population shown in FIG. 7C for flowering times, plant heights, grain filling period, and days to senescence.

FIG. 8 illustrates example variance component results for temporal vegetation indices derived from example orthomosaics.

FIG. 9 illustrates an example plot of temporal correlation between temporal values of all vegetation indices and senescence.

FIG. 10 illustrates example temporal correlations between example temporal values of vegetation indices.

FIG. 11 illustrates example quantitative trait loci linked to grain filling period, days to senescence, flowering times, and plant heights.

FIG. 12 illustrates example prediction abilities for multivariate prediction of temporal senescence and example univariate prediction of grain filling period, according to implementations of the present disclosure.

FIG. 13 illustrates an example correlogram between traits of an entire hybrid population and the top yielding ten percent, illustrating an example of the relationship between grain filling period and grain yield.

FIG. 14 illustrates an example correlogram between traits for recombinant-inbred-line populations in irrigated and non irrigated trials.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The term “artificial intelligence” is defined herein to include any technique that enables one or more computing devices or computing systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

A phenotype is an observable characteristic of an organism (e.g., a plant or animal) of functional, biological or economic interest. Phenotypes can include a wide range of features, including physical attributes, behavioral traits, and physiological functions (e.g., metabolism, disease resistance). Phenotypes are the observable endpoints of complex interactions between an organisms genes and the environment. Thus, observing the phenotype is an efficient way to select organism for breeding, predict the behavior or performance of organisms, and otherwise understand the organism.

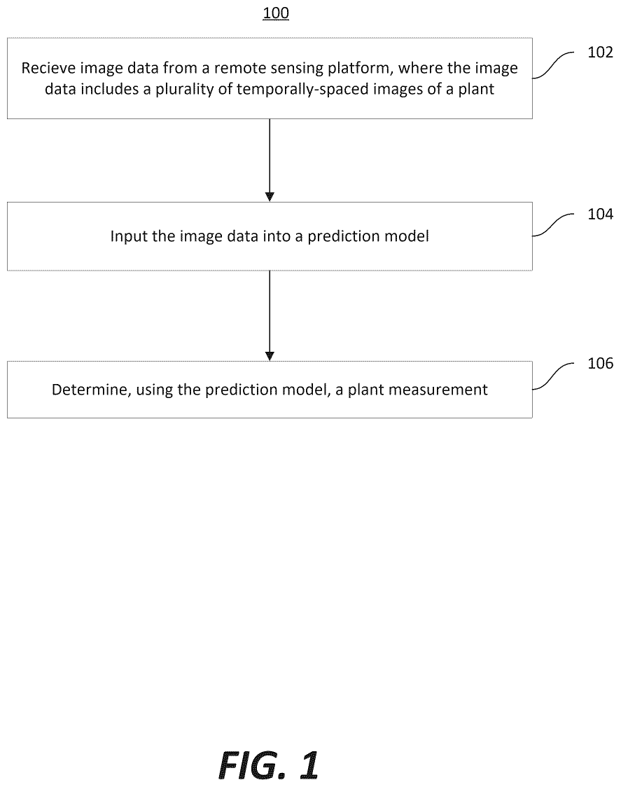

With reference to FIG. 1, implementations of the present disclosure include methods of determining phenotypic measurements based on image data. At step 102, the method includes receiving image data from a remote sensing platform. In some implementations, the remote sensing platform can include an unoccupied aerial system (UAS). Optionally, the remote sensing platform comprises a plurality of UASs (e.g., a group of drones), ground vehicles, human or animal carried sensors, or satellites. Yet more examples of remote sensing platforms include robots, and handheld sensors.

The image data can include a plurality of temporally-spaced images of a plant. In some implementations, the image data can be collected from an altitude of less than less than or equal to 100 meters. Alternatively or additionally, the image data can include images with a resolution greater than or equal to 1 cm per pixel (i.e., the resolution can be high enough that each pixel corresponds to a single square centimeter, or even higher, so that one pixel corresponds to less than a single square centimeter. As yet another example, the image data can include images with a resolution greater than or equal to 25 cm per pixel. Alternatively or additionally, the image data can be collected at altitudes of less than or equal to 100 meters.

At step 104, the method can include inputting the image data into a prediction model to get individual timepoint measures of senescence or stay green. In some implementations, the prediction model includes a phenomic prediction model. Alternatively or additionally, the prediction model can include a genomic prediction model. Alternatively or additionally, the prediction model can include metabolomic, and/or or proteomic prediction models. The present disclosure contemplates that combinations of phenomic, genomic and other -omic prediction models can be used in some implementations of the present disclosure.

Alternatively or additionally, the prediction model is a trained machine learning model.

At step 106, the method can include determining, using the prediction model, a phenotypic measurement. Non-limiting examples of phenotypic measurements include plant senescence or stay green. In some implementations of the present disclosure, the method can further include determining a dependent phenotypic trait of interest based on the phenotypic measurement. Non-limiting examples of a dependent phenotypic trait of interest are grain filling period and yield.

In some implementations, the method can further include selecting varieties based on the phenomic prediction of senescence and stay green.

In some implementations, the method can further include determining a senescence score based on the image data.

In some implementations of the present disclosure, the method can further include controlling the remote sensing platform based on the phenotypic measurement. For example, controlling the remote sensing platform can include changing the frequency that imaging is performed using the imaging device, and/or causing the remote sensing platform to acquire images of specific organisms or groups of organisms (e.g., choosing which field of crops should be imaged next).

In some implementations of the present disclosure, the method can further include controlling agricultural equipment based on the phenotypic measurement. For example, if the phenotypic measurement or prediction is plant senescence, the method can include configuring the agricultural equipment to stop delivering nutrients or water to the crops. As another example, the method can include harvesting the crops based on the detection that plant senescence has begun, or that a certain number of days have elapsed from in the grain filling period, or that plant senescence is predicted to occur a certain number of development units in the future.

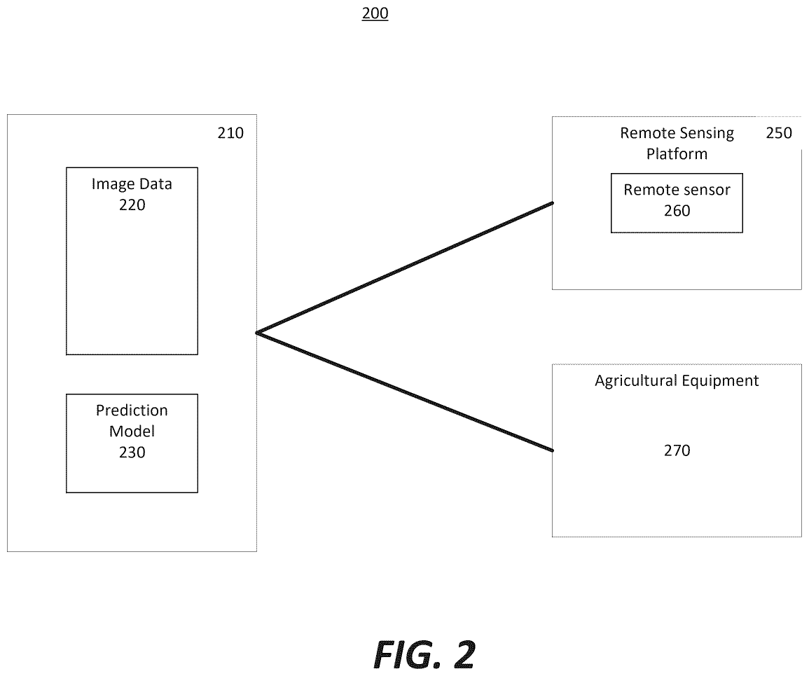

Implementations of the present disclosure include systems for predicting phenotypic measurements using prediction models. An example system 200 is shown in FIG. 2. The system 200 can include a remote sensing platform 250. The remote sensing platform 250 can include a remote sensor 260. As used herein, a remote sensor 260 can be an RGB camera/sensor, multispectral camera, hyperspectral camera, LIDAR, thermal camera, or point sensor. It should be understood that the remote sensor 260 can include any number of pixels and any resolution. The remote sensor 260 can be configured to capture image data. Optionally, the image data can include temporally-spaced images of a plant, or a group of plants (e.g., a field of crops).

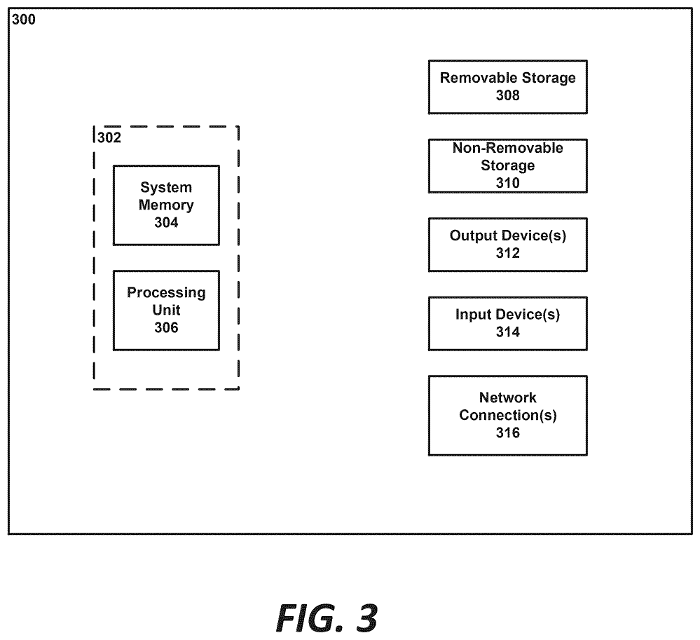

The system 200 can further include a computing device 210 coupled to the remote sensing platform. The computing device 210 can include any or all of the components shown and described with reference to the computing device 300 shown in FIG. 3. The computing device 210 can be operably connected to the remote sensing platform 250 (e.g., by a wireless or wired network). In some implementations of the present disclosure, the system 200 can include more than one remote sensing platform 250 (e.g., a group of drones, satellite constellation) and/or more than one computing device 210 (e.g., a server), where the computing devices and remote sensing platforms 250 are networked together to implement the methods 100 described with reference to FIG. 1. For example, the computing device 210 can be configured to store image data 220 and input the image data 220 into a prediction model, and/or determine, using a prediction model 230 stored on the computing device 210, a phenotypic measurement.

In some implementations of the present disclosure, computing device 210 can be configured to control the remote sensing platform 250. For example, the remote sensing platform 250 can be controlled remote based on the relationship between the phenotypic measurement and the image data. Controlling the remote sensing platform 250 can optionally include changing the frequency that imaging is performed using the remote sensor 260, and/or causing the remote sensing platform 250 to acquire images of specific organisms or groups of organisms (e.g., choosing which field of crops should be imaged next).

The system 200 can optionally further include agricultural equipment 270. Non-limiting examples of agricultural equipment 270 include systems that harvest crops, irrigate crops, and fertilize crops. In some implementations, the agricultural equipment 270 can be controlled based on the phenotypic measurement. For example, if the agricultural equipment 270 includes an irrigation, the sprinkler can be controlled to stop irrigation to plants based on when plant senescence is measured or predicted to occur in those plants.

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Examples of tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

A study was performed of an example implementation of the present disclosure The example implementation of the present disclosure was studied to detect plant senescence, stay green, grain filling period and estimate yield. Plant senescence here is the death of annual plants and represents the end of the grain filling period. The grain filling period is the period of time between plant flowering and senescence which is when all nutrients are moved into the grain. Senescence can be estimated from unoccupied aerial systems (also referred to herein as “UAS” or “drones”) using remotely sensed imaging by evaluating spectral reflectance, vegetation indices calculated from this reflectance and/or structural features. This can be done for each image visually, using a phenomic prediction model based on spectral (e.g. vegetation indices) or structural features, or using artificial intelligence. A mathematical or statistical model (e.g. mechanistic growth fit model, and/or a Weibull fit model) can be fit to these features with respect to growth stage over time to interpolate the amount of senescence that has occurred between imaging dates. This allows the date of a specific amount of senescence to be identified and thus objectively compared between different varieties or environments. Flowering can be estimated similarly to senescence. Subtracting the flowering date from the senescence date objectively calculates the grain filling period. The measurement of grain filling period is advantageous because it is often highly correlated with grain yield, an important economic trait, especially in stress environments. Furthermore, individuals with deviations between grain filling period and grain yield can suggest useful genetic mechanisms and genes such as those for improving grain yield. This approach also eliminates confounding by late flowering. Senescence and grain filling periods are useful phenotypic measures for plant breeders to compare varieties for yield potential and aid in selection, especially under stress. This is also useful for yield estimation before harvest, which is of value to crop modelers, and biologists to identify biological processes, and pathologists and entomologists to identify the effects of disease and pests. It is also of use to farmers, governments and grain traders where the price of grain is dependent on supply and demand and supply is difficult and inaccurate to measure, so improved prediction of yield and grain supply earlier and more accurately can create economic advantages.

“Full season” varieties, with longer grower periods tend to yield more; however farmers want to get their crops out of the field as soon as possible or in some locations winter will kill the crop before grain is finished filling. The reason certain plants senesce late may be because they flower late which does not necessarily increase yield, or because they have a longer grain filling period. It is also well known that early senescence and death, such as due to drought stress or disease, substantially reduces yield. Both grain filling period and senescence are only partial and imperfect information and also have been very difficult, time consuming and subjective to estimate visually. Use of remote sensing approaches can inexpensively, quickly and objectively estimate senescence, flowering time and grain filling period. Scaling this would be valuable for plant breeders to select varieties and for other professions as explained above to have objective phenotypic measurements and predictions. As another example, plant biologists looking to sample RNA at a certain stage in plant senescence/death have not yet had an objective method to compare stages and this method provides that objective phenotype.

Alternatively or additionally, the systems and methods of the example implementation can be used for plant breeding. For example, implementations of the present disclosure can be used with remote sensing pipelines to identify plants with longer grain filling periods but shorter seasons, these would have better yield but also be harvestable by farmers more quickly. Another example application of the example implementation is to identify genetics that are anomalies and have high yield despite short grain filling periods.

Alternatively or additionally, the example implementation can be used for crop physiology and modeling—to use in making objective development standards after flowering. This can be the way that the important V1 through VT stages are used before flowering, but after flowering there is no known method beyond the inaccurate and not biologically meaningful “days after VT”.

Alternatively or additionally, implementations of the present disclosure can be used for to use in satellite imagery estimation of crop yield and condition. This knowledge affects global grain prices, and has practical implications for organizations including governments, grain traders, satellite data providers, etc.

Alternatively or additionally, implementations of the present disclosure can be used by farmers to determine when no more inputs should be provided to a field or crop. At or after senescence has started, plants may not uptake any additional inputs (e.g., fertilizer, water), so inputs that are provided to a crop after the start of senescence are wasted.

Plant senescence, the drying down and death of a plant, is an important phenotype in maize related to grain filling and yield. This is the opposite of stay-green, a desirable but difficult to measure phenotype. Advances in sensing technologies and image analysis have enabled the collection of high-throughput phenomic data in the field that can quantify challenging phenotypes. These tools have led to the emergence of a new paradigm in predictive plant breeding, where large-scale phenomic data may be combined with genomic, environmental or other information to predict plant performance under different environmental conditions. In the present study, 515 recombinant inbred lines (RILs) were developed using three bi-parental populations and grown in irrigated and non-irrigated trials. The RILs were previously genotyped and here phenotyped by unmanned aerial system (UAS: drone with RGB sensor) with 14 flights across growth. Temporal (longitudinal or 4D) senescence was scored screening plots in the last four flights in both trials and subjected to a mechanistic growth fit model (R\hat{ }2=0.98−| and 0.97 in irrigated and non-irrigated trials) allowing interpolation of days data was not collected and forward prediction of senescence progress. Days to senescence (DTSE) and grain filling period (GFP) were then quantified as two novel traits then shown to be related to grain yield in maize. Phenomic (32 vegetative indicies×14 flights) and genomic (11,334 markers) data from 515 RILs, alone and together, were used to predict GFP and temporal senescence via univariate and multivariate prediction respectively across trials. Results showed that phenomic prediction outperformed the genomic prediction in predicting GFP and temporal senescence in the most challenging prediction scenarios (e.g., untested genotypes in observed and unobserved environments). Quantitative trait loci (QTLs) suggested a candidate gene (hb45) for GFP as well as DTSE and temporal senescence involving homeobox-leucine zipper (HB-Zip) family of transcription factors that influence kernel formation dimension in maize. Overall, this study demonstrated how large amounts of high-quality phenomic features can lead to novel phenotypes that improve efficiency and accuracy of predictive plant breeding and help better understand changes in plant biology across growth.

Plant aging refers to a biological period from the slowing to cessation of plant cell divisions during reproductive growth stages. Senescence occurs at the late terminal period of the plant aging process in annual plants and most crops, eventually resulting in phenotypically degenerative senescent tissues in plant organs (Lim et al., 2003). Perennial plants and trees experience senescence differently as preparing for dormancy. Senescence is manipulated by endogenous and exogenous triggers that promote plant fitness and adaption to fluctuating environments (Großkinsky et al., 2017; Sade et al., 2017; Woo et al., 2013). Several transcription factors including NAC, WRKY, MYB, AP2/EREBP, bZIP, and bHLH have been attributed to regulation of senescence in maize (Lauter et al., 2005; Wu et al., 2016; Young et al., 2004), wheat (Chapman et al., 2021; Gregersen and Holm, 2007), gardenia (Tsanakas et al., 2014), rice (Liu et al., 2008) and Arabidopsis (Gepstein et al., 2003; Lim et al., 2003). Epigenetic regulations including chromatin remodeling, DNA and histone modification also involve the regulation of senescence (Yolcu et al., 2017). Abiotic triggers hasten stress induced senescence (Sade et al., 2018). For example, drought driven senescence was manipulated in maize (Young et al., 2004), tobacco (Rivero et al., 2009), rice (Raineri et al., 2015) and Arabidopsis (Lee et al., 2012). Dark-induced senescence was manipulated in Arabidopsis (Sakuraba et al., 2014) and rice (Fukao et al., 2012). Salinity related senescence was manipulated in Arabidopsis (Balazadeh et al., 2010), wheat (Yang et al., 2003; Zheng et al., 2008) and tomato (Ghanem et al., 2008). Various strategies of hormone hemostasis were reported that regulate the senescence, and of those, cytokinin and ethylene functions have been the best described (Schippers et al., 2007) while other hormones could have different actions on senescence (Schippers et al., 2007; Schippers et al., 2015). Even though regulatory mechanisms of senescence were examined comprehensively at molecular, biochemical, and physiological levels in Arabidopsis (Lim et al., 2003), the complexity of senescence and its relations with phenological and other complex traits are diverse and need further investigations with both advanced phenotyping techniques and other model organisms. Senescence occurs progressively over time and reveals variation among different genotypes (Pommel et al., 2006).

In maize, the response of senescence to source-sink ratios varies by hybrid, with short maturity hybrids' grain yield often being restricted by the robustness of reproductive sinks during the grain filling period compared with long maturity hybrids (Capristo et al., 2007).

Besides being a biological process, senescence can play a major functional role in crop productivity (Abeledo et al., 2020; Großkinsky et al., 2017; Sade et al., 2018). As a byproduct of modernization of maize breeding, more efficient usage of assimilates and their transportation to grain during reproductive stage has been associated with greater grain yield in maize (Ciampitti and Vyn, 2013). An early onset of senescence is accelerated by stress conditions, which affect source-strength adversely due to degradation of photosynthetic activity caused by photosynthesis feedback inhibition in source leaves. (Albacete et al., 2014).

Thereby remobilization of assimilate from source to sink organs is interrupted, which is a limiting factor for increasing yield, especially in annual crops (Pérez-Alfocea et al., 2010). Postponing senescence promotes the stay green behavior has been selected because it can be demonstrated to increase yield (Abeledo et al., 2020; Chapman et al., 2021; Fu et al., 2011; Messmer et al., 2011). Overall, most studies underline the impact of the longer duration of photosynthetic capacity during grain filling period (GFP) in reproductive stages to achieve greater grain yield in maize (Gregersen et al., 2013); that phenomenon can be better scrutinized by evaluating the source-sink strengths, senescence and perceived stay green in a temporal manner.

Measurement of senescence of previous studies has largely been subjective and qualitative based on one or few timepoints. Objectively capturing the temporal (longitudinal or 4 dimensional) quantitative variation of senescence is infeasible to impossible by visually rating across each plot in field studies; being time-consuming and low in scale and temporal dimensions. In contrast, automated field-based high throughput phenotyping (FHTP) using remote sensing techniques from the beginning to the end of senescence, is practical to quantify senescence over time, as well to investigate relationships with other complex traits including GFP and yield.

Unoccupied aerial system (e.g., drones equipped with sensors) can be flown across multiple growth stages and provide high resolution remotely sensed images where temporal patterns of growth and senescence acceleration are captured. High resolution images (e.g., low flight elevations) contain multiple reflectance bands (e.g., 400-700 nm by RGB; 400-1000 nm by multispectral sensors) directly related to photosynthetic pigments sensitive to the visible spectrum (400-700 nm) (Jacquemoud and Baret, 1990). Temporal reflectance values specific to each genotype can be utilized quantitatively as a merit of temporal variation of photosynthetic activity and senescence related to plant fitness. In addition, the GFP can also be quantified as a period between days to silking and senescence. In the current literature, senescence in wheat (Hassan et al., 2021) and maize (DeSalvio et al., 2022; Makanza et al., 2018) was quantified via FHTP; senescence-related loci were discovered in wheat (Hassan et al., 2021), while no senescence-related loci have yet been reported in maize via FHTP.

In general, FHTP involves predictive plant breeding and improved precision in predicting agronomically important complex traits in crop plants through either univariate or multivariate predictions (Araus and Cairns, 2014; Araus et al., 2018; Herr et al.; Shi et al., 2016). Several reports across species have shown prediction of yield related traits (e.g. grain yield in maize and biomass in rye, but excluding senescence or stay green) were enhanced using the measures collected from FHTP in multivariate or univariate prediction models (Crain et al., 2018; Galán et al., 2020, 2021; Juliana et al., 2019; Krause et al., 2019; Krause et al., 2020; Rutkoski et al., 2016; Sun et al., 2019; Sun et al., 2017). Phenomic data with high number of heritable phenomic features collected from FHTP with a high time dimension was shown to be equal to or could outperform the genomic prediction of grain yield in maize conducted across diverse trials (Adak et al., 2022). Yet inconsistencies in prediction ability have been reported across different years and trials (Adak et al., 2022; Adak et al., 2021; Crain et al., 2018; Galán et al., 2021; Rutkoski et al., 2016). This might be due to fewer time dimensions, fewer phenomic features from FHTP, or both, leading to insufficiency in capturing environmental characteristics; we would expect this to cause inconsistency in the prediction ability of models. Interactions between reflectance bands and environments was shown to play a greater role in obtaining consistent prediction abilities across diverse environments than genomic-environment interactions (Montesinos-López et al., 2017).

In this research, a FHTP UAS platform was utilized including the RGB sensor with greater resolution (e.g. greater pixels per plot due to lower flight elevation and time dimension additional flight times) than previous studies to quantify senescence, GFP, as well as plant growth for two different maize populations of recombinant inbred lines (RILs) and a diverse hybrid population grown under different management conditions (optimal and late planting trials for hybrids; drought and irrigated trials for RILs). In this study we aimed to (i) quantify the temporal senescence, stay green and GFP via high resolution images temporally across different trials and populations using a new model and discover vegetation indices with high correlations to senescence and GFP scores, (ii) discover quantitative trait loci (QTLs) linked to senescence and GFP, (iii) conduct genomic and phenomic prediction for GFP and temporal senescence progression. Ultimately helping to understand the genetic architecture of these phenotypes and gain insight into the missing heritability.

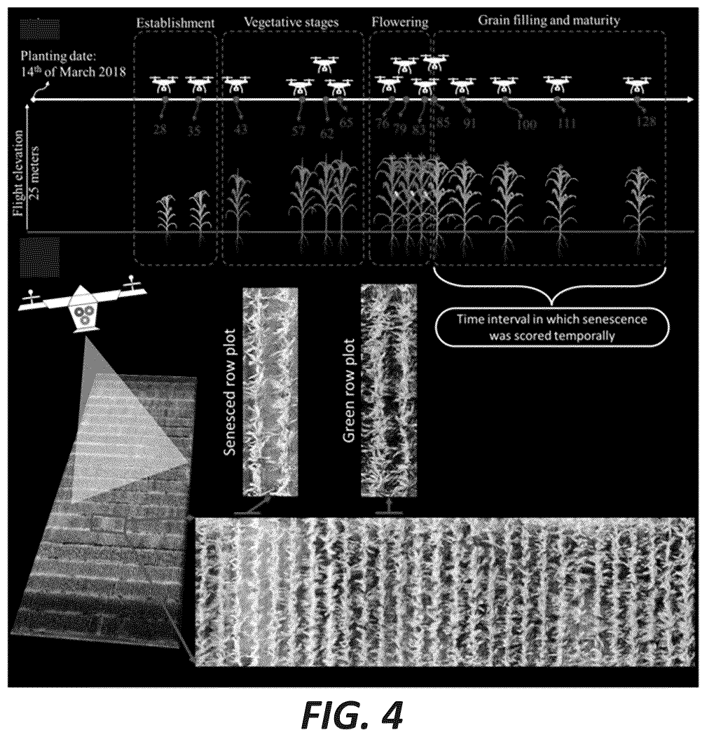

In the study, three different bi-parental populations were used to develop recombinant inbred lines (RILs) used in this study; their parental lines were selected based on segregating loci controlling plant height and grain yield discovered in a previous genome-wide association study, unrelated to stay green, senescence or grain filling period (Chen, 2016; Farfan et al., 2015). 515 RILs were developed from the crosses of Ki3/NC356 (tropical/tropical; 238 RILs), LH82/LAMA (temperate/tropical; 176 RILs) and Tx740/NC356 (tropical/tropical; 101 RILs). All mapping populations were grown in two different management conditions, namely irrigated and non-irrigated trials with the planting date of 14th of March in 2018. In each trial, RILs were grown according to randomized complete block design (RCBD) with two replications that were constructed by a range (horizontal grid) and row (lateral grid) spatial pattern. 32 ranges were used for all three biparental populations while 17, 13 and 8 rows were used for Ki3xNC356, LH82xLAMA and Tx740xNC356 populations in both trials. Plots were 3.81 m long and distance between plots was 0.76 m. Unoccupied aerial flights, image assembly and phenomic data extraction were performed. The unoccupied aerial system (UAS) was a DJI Phantom 3 Professional (rotary wing drone) equipped with an RGB camera (12-megapixel DJI FC300X). The UAS was flown at an altitude of 25 meters with 80% forward and side image overlap accuracy in both trials (irrigated and non-irrigated) and achieved a resolution of ˜1 cm per pixel. The UAS was flown 14 times and each flight covered both trials at each flight date, and flight date was reported as days after plating (DAP) throughout the manuscript (FIG. 4). Raw drone images (.jpeg) were assembled in 150 Pix4Dmapper software (https://www.pix4d.com/) to create orthomosaic (.tiff) for each flight.

Various vegetation indices (VIs) were extracted from each row plot in orthomosaics. To do so, first UAStools were used to create the shapefiles covering each row plot using their range and row numbers and plot names as it is seen in UAS Tools (Anderson 154 and 2020). Second, modified FIELDimageR (https://github.com/OpenDroneMap/FIELDimageR) steps with shape files from the first step were used to extract the VIs (Matias et al., 2020); The same scale to score senescence was used in this study as follows: (i) orthomosaics belonging to 85, 91, 100, 111, and 128 DAPs were visualized in QGIS along with shape files (FIG. 4), (ii) each shape file covering each plot was visually checked to ensure they covered the row plots correctly, shape files were dragged slightly if necessary to cover row plots accurately and (iii) third, each row plot within their shape file were then scanned and scored between 0 (“green” labeled plot shown in FIG. 4) and 100 (senesced plot shown in FIG. 4) in irrigated and non-irrigated trials using the orthomosaic belonging to 85, 91, 100, 111, and 128 DAPs; senesce started at 85 days after planting. Optionally, the scanning and/or scoring can be performed using any of the machine learning or artificial intelligence systems and methods described herein.

After completing temporal senescence scoring, these scores for each row plot were fit using mechanistic growth model in irrigated and non-irrigated trials as follows:

$$\begin{matrix}Equation 1 & \\ y = a * ( 1 - b * exp ( - c * Flight ) ) ; & Eq 1\end{matrix}$$

where y contains temporal senescence scores of each row plot derived from orthomosaics belonging to 85, 91, 100, 111, and 128 DAPs, a is asymptote, b is scale, c is growth rate, and Flight are the numbers of 85, 91, 100, 111, and 128, which correspond to DAPs of flight dates where senescence was scored (FIG. 4).

After a mechanistic growth model was developed for each row plot in both trials, days to senescence time as days after planting were calculated for each row plot and denoted as DTSE as follows:

$$\begin{matrix}Equation 2 & \\ Days to senescence ( DTSE ) = - \frac{ln ( \frac{a - y}{a * b} )}{c} ; & Eq2\end{matrix}$$

where each variable is the same as the variables in mechanistic growth model. Here, however, y was chosen to be a senescence score of (85%) for each row plot, and then DTSE was calculated using asymptote (a), scale (b), and growth rate (c) information for each row plot in both trials. The value of 85% was chosen as the default cutoff based on the consistency of this measure that we visually observed, any number between 80% and 90% appeared similar.

The days to anthesis (DTA) and silking (DTS) were recorded as DAPs for each row plot when the tassel and silk appeared through half of the plants in the plot.

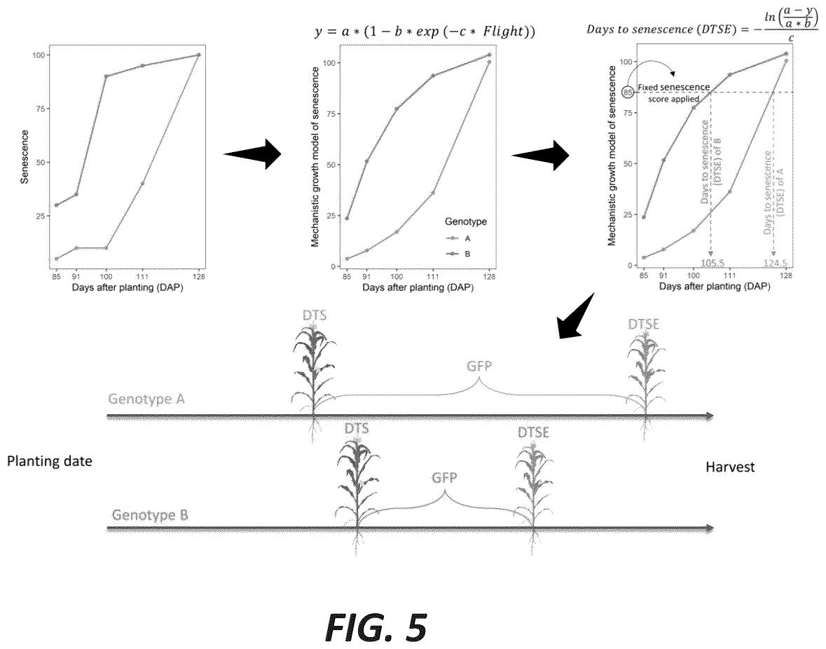

Grain filling period (GFP) was calculated between days to senescence (DTSE) and silking (DTS) for each row plot in both trials (irrigated and non-irrigated) as follows:

$$\begin{matrix}Equation 3 & \\ GFP = DTSE - DTS ; & Eq3\end{matrix}$$

Where GFP is the grain filling period of each row plot, DTSE and DTS are days to senescence and days to silking of each row plot respectively. Illustration of (A) raw senescence scores, (B) developing of mechanistic growth model for senescence, (C) determining days to senescence (DTSE), and (D) calculating of grain filling period (GFP) are presented in FIG. 5.

Three different terminal plant heights were measured from each row plot manually at the end of plant growth once: from ground to tip of tassel (PHT), to collar of flag leaf (FHT) and shank of first ear (EHT).

Statistical analysis of temporal data each RIL for each VI as follows:

$$y = μ + G + F + M + GF + GM + FM + GFM + range + row + replication ( M ) .$$

Where u is the grand mean; y is the temporal value of each VI belonging to 14 flight date for each plot (Docketing <Docketing@mcciplaw.com>); G is genotype effect of the 515 RILs; M is management effect of irrigated and non-irrigated trials; F is flight effect of 14 drone flights as days after planting units as shown in FIG. 4. GF, GM and FM are the two-way and GFM is the three-way interaction terms in the model. Spatial variation was corrected by range, row and replication effects; replication was treated nested within M since each trial was planted with two replications where RIL row plots were randomized in each replication and trial.

$$\begin{matrix}Equation 5 & \\ Temporal repeatability = \frac{{σ}_{G}^{2}}{{σ}_{G + \frac{{σ}_{GF}^{2}}{k} + \frac{{σ}_{GM}^{2}}{l} + \frac{{σ}_{GFM}^{2}}{k \times l} + \frac{{σ}_{ε}^{2}}{k \times l \times r}}} ; & Eq 5\end{matrix}$$

Temporal repeatability was calculated the using the results of R, OGM, OGFM and og components calculated in Eq 4. The k, l and r are the numbers of flights, managements, and replications respectively.

Temporal senescence scores fit by mechanistic growth model for each row plot (Eq. 1) were also subjected to Eq. 4 to predict the temporal genotypic values of each RIL. Every component is same as the Eq. 4 except for F. F contains the last four flights as DAP (85, 91, 100, 111 and 128 234 DAPs) where senescence was scored temporally.

Eq. 4 was also run to predict the genotypic value for flowering times (DTA and DTS), plant heights (PHT, FHT and EHT) as well as DTSE and GFP without the F component and it's interactions, since they were recorded and/or calculated based on single time point. Repeatability was also calculated for single time point derived traits using the Eq. 5 without the F component or it's interactions included.

The Pearson's correlation was calculated between temporal senescence (at 85, 91, 100, 111 and 241 128), DTA, DTS, PHT, FHT, EHT, GFP and DTSE in irrigated and non-irrigated trials separately.

In addition to the 515 RILs, an unrelated set of 280 unique maize breeding hybrids were also used in this study to evaluate the correlation with grain yield, which has minimal relevance in inbreds. These hybrids were planted Mar. 3, 2017 based on randomized complete block design with two replications. Flowering times (DTA and DTS) and plant heights (PHT, FHT and EHT) were recorded as explained above, grain yield was recorded for each row plot via combine harvester and converted to t/ha. The hybrid population was flown 23 times with a DJI Phantom 3 Professional with a 12-megapixel DJI FC300X camera at an altitude of 25 meters above the ground. The last four flights corresponding to 105, 118, 133 and 146 DAPs were used to score senescence, and then DTSE and GFP were calculated for the hybrid population as explained in FIG. 5. Equation 4 without management effects were applied to hybrid population for temporal senescence scores. Equation 4 without flight and management effects were also applied for flowering and plant height traits and grain yield. The Pearson's correlation between all traits were calculated using all hybrids and top ten yielding hybrids, especially to highlight the correlation between GFP and grain yield. Genomic DNA extraction was described in (Chen, 2016), and is summarized here. Eight seedlings of each RIL were bulked and a modified CTAB protocol was used to extract the genomic DNA of each RIL (Chen and Ronald, 1999). Each RIL was then genotyped according to the Infinium whole-genome genotyping analysis, yielding 17,444 genome-wide single nucleotide polymorphisms (SNPs) across ten chromosomes with the physical location information of B73 RefGEN_v3 (Schnable et al., 2009).

Several steps were completed before QTL mapping. First SNPs and RILs with greater than 10 percent missing calls were filtered out, and 5,316, 5,628, and 6,231 polymorphic SNPs remained for the Ki3xNC356, Tx740xNC356, and LH82xLAMA populations, respectively. Second, SNPbinner (v.0.1.1) was used to correct double recombinants events in genomic data, in which p (emission probability), -r (continuous genotype region) and -c (transition probability) were set to 0.9, 0.1% of the chromosome size and crosscount of 7,500,000 respectively (Gonda et al., 2019). Third, corrected genomic data by SNPbinner was then opened with QTL IciMapping (Meng et al., 2015) to remove the redundant markers using the BIN function. Fourth, linkage maps were constructed using “By Anchor Only” setting, and polymorphic SNPs were ordered according to their physical locations using the “By Input” algorithm.

After filtering, correcting double crosses and removing the redundant SNPs, 1,530, 2,571, and 2,324 SNPs were used to construct linkage maps for Tx740xNC356, Ki3xNC356, and LH82xLAMA populations, respectively in IciMapping software. The linkage maps were 1,315, 1,207, and 1,474 cM in length for Tx740xNC356, Ki3xNC356, and LH82xLAMA populations, respectively; calculated according to the Kosambi mapping function. QTL mapping was applied using inclusive composite interval mapping (Li et al., 2007) in IciMapping software, and was run for flowering times, plant heights, temporal senescence scores and GFP traits of each bi-parental population using “BIP” function. “POP.ID=4” was set for RIL population, and “ICIM-ADD” was selected to run additive QTL model in IciMapping software.

The example implementation further included systems and methods for genomic and phenomic biomarker prediction.

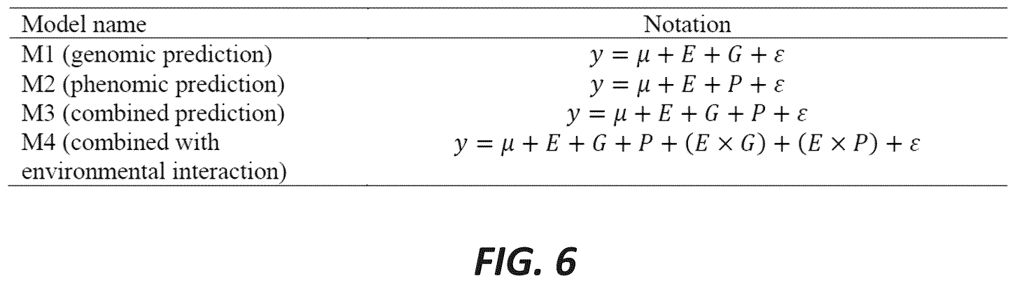

A univariate prediction was applied to predict GFP using both genomic and phenomic biomarker data using BGLR package in R (Pérez and de los Campos, 2014). To do so, four prediction models were used (FIG. 6). A genomic relationship matrix was constructed using genomic data with 11,334 SNPs of 515 RILs (VanRaden, 2008). Unlike genomic data the phenomic biomarker data is unique to the irrigated and non-irrigated trials and each phenomic data set contained the 32 VIs belonging to 14 flight times across the 515 RILs; phenomic data of the two trials was first merged and then a phenomic biomarker relationship matrix was constructed in a similar way as the genomic relationship matrix. In the prediction models, y is the GFP value of each RILs in irrigated and non-irrigated trials; E is the environment matrix designed for irrigated and non-irrigated trials and constructed as follows: with

$$E ∼ N ( 0 , \frac{{Z}_{E} {Z}_{E}^{′}}{2} )$$

Z_{E} as the incidence matrix that connected the phenotypes with environments (irrigated and non-irrigated trials); G was the vector of genomic effects and was modeled using the matrix of SNP markers as linear combinations between p marker and their corresponding effects such that g={gᵢ=Eⱼ₌^{p}xᵢⱼbⱼ}˜N(0, Z_{g}GZ′_{g}σ_{g}²) with

$$G = \frac{{MM}^{′}}{p} ,$$

M is the centered and standardized (by columns) matrix of marker SNPs, σ_{g}² was the associated variance component, and Z_{g} was the corresponding incidence matrix that connected phenotypic observations with genotypes. P is the phenomic biomarker relationship matrix and designed as follows:

$$P ∼ N ( 0 , \frac{{RR}^{′} {σ}_{r}^{2}}{14 \times 32} )$$

where R was relationship matrix derived from RGB UAS temporal phenomic biomarker data, and σᵣ² was the associated variance component. The interaction terms between environments E and the genomic markers (E×G) or the phenomic data (E×P) according to 304 (Jarquín et al., 2014). Briefly, the Hadamard product (#cell-by-cell product) of the associated 305 co-variance structures of these model terms was computed such that

$$( E \times 306 G ) ∼ N ( 0 , \frac{{Z}_{E} {Z}_{E}^{′} \# {Z}_{g} {GZ}_{g}^{′} {σ}_{g \times E}^{2}}{2} )$$

$$and$$

$$( E \times P ) ∼ N ( 0 , \frac{{RR}^{′} \# {Z}_{E} {Z}_{E}^{′} {σ}_{r \times E}^{2}}{14 \times 32 \times 2} )$$

with σ_{g×E}², and σ_{r×E}², as the 307 corresponding variance components.

A multivariate/multitrait model was also applied to predict the temporal senescence using the same prediction models (FIG. 6) in BGLR in R (Pérez-Rodríguez and de los Campos, 2022). Here y is the senescence values of each RILs belonging to 85, 91, 100, 111 and 128 DAPs in irrigated and non-irrigated trials.

Each prediction model was trained using 70% of RILs (360 RILs) while the remaining 30% of RILs (155 RILs) was used as a held-out data set. So, 70% RILs was considered as tested RILs since they were used in training the models while 30% of RILs were considered as untested RILs since they were excluded in training the models. Each model was run 50 times, tested and untested plants were randomly selected in each.

To calculate the prediction ability of the prediction models, Pearson's correlation was calculated between predicted and actual values of temporal senescence and GFP in each of 50 repeats. One correlation was calculated for GFP (univariate) while four correlations were calculated for temporal senescence since four senescence scores at 85, 91, 100, 111 and 128 DAPs (multivariate) were used in each repeat. Then, the mean and standard deviations of the correlations from 50 replicates were declared for GFP and temporal senescence as predictive ability in four cross-validation prediction scenarios and each model.

Four cross-validation prediction scenarios, namely CV1, CV2, CV3, and CV4, were applied to each prediction model. In the CV1 and CV2 prediction scenario, the models were trained using the tested RILs in both the irrigated and drought trials, and then the tested and untested RILs in both trials were predicted. Next, the prediction abilities were calculated for the tested and untested RILs corresponding to CV1 and CV2. In CV1 and CV2, tested RILs from both trials were used in training the models so both trials were considered as observed environments. In other words, CV1 and CV2 refer to prediction abilities of tested and untested genotypes in observed environments respectively.

In the CV3 and CV4 prediction scenario, the models were trained using the tested RILs in only the irrigated trial, and then the tested and untested RILs in only drought trial were predicted.

Next, the prediction abilities were calculated for the tested and untested RILs corresponding to CV3 and CV4. In CV3 and CV4, tested RILs from only irrigated trials were used in training the models. So the irrigated trial was considered as an observed environment while drought trial was considered as an unobserved environment. In other words, CV3 and CV4 refer to prediction abilities of tested and untested genotypes in the unobserved environment respectively. Temporal progression of senescence recorded across 85, 91, 100, 111 and 128 DAPs was explained most by the flight effect (F: 88.8%) and temporal repeatability was computed as ˜0.56. Flowering times (DTA and DTS) had repeatability of ˜0.92; height related traits (EHT, 351 FHT and PHT) had repeatability of ˜0.65, ˜0.76 and ˜0.78. Grain filling period (GFP) had 352 repeatability of 0.63 and days to 85% senescence (DTSE) as modeled had repeatability of 0.67.

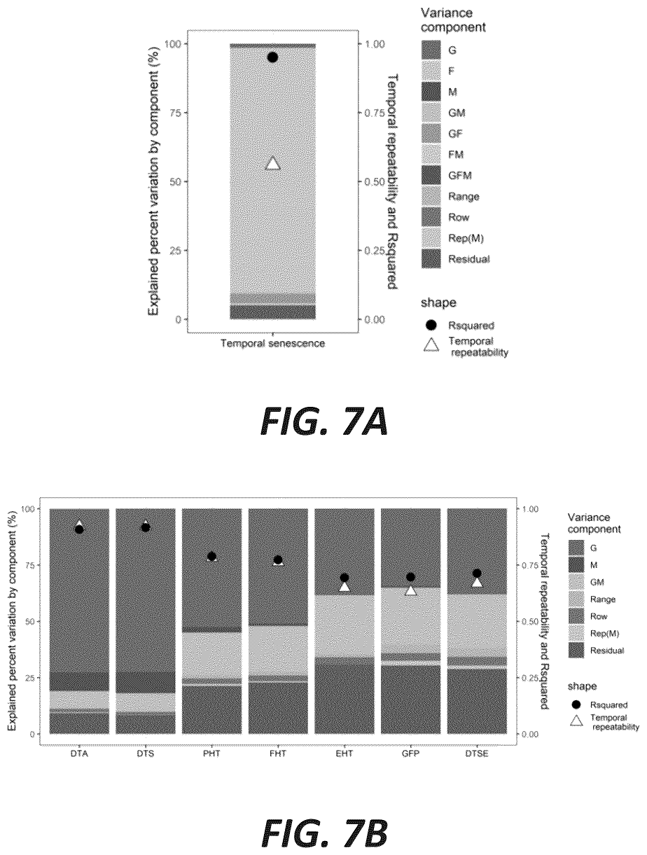

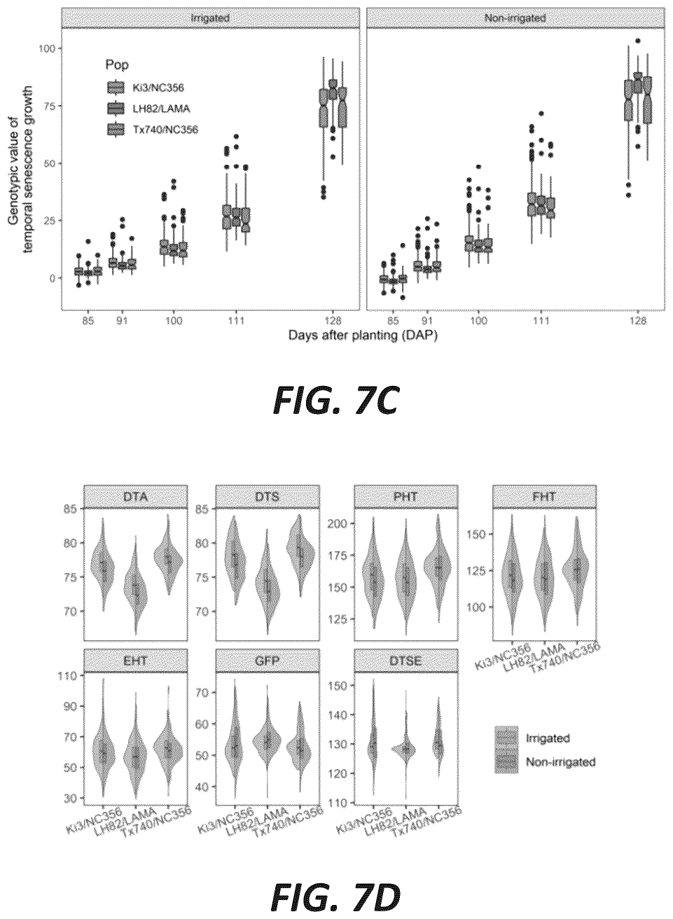

Overall, the pedigree effect (G) explained the highest percent of variation for plant heights traits (EHT, FHT and PHT), flowering traits (DTA, DTS) and DTSE and GFP between 34.8 to 72.6% (FIG. 7A and FIG. 7B).

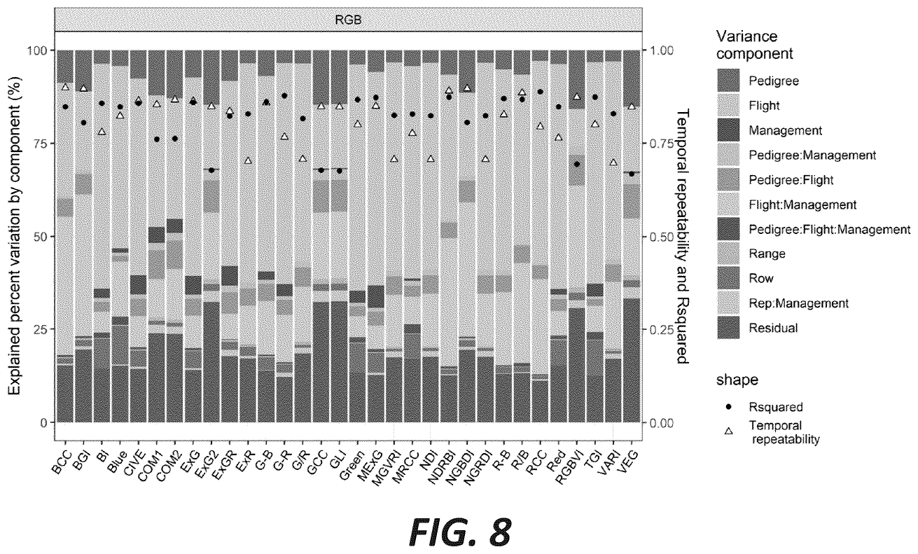

Variance component results of VIs resulted in flight effect (F) and flight-management 357 interactions (FM) explaining the majority of total variation in overall vegetation indices (FIG. 8). Temporal repeatability's were found to change between 0.67 and 0.89 depending on the VIs (FIG. 8).

Genotypic values of traits across irrigated and non-irrigated trials were studied.

Temporal senescence increased dramatically from 85 to 128 days after planting; senescence progression followed −0.8±2.3, 4.8±3.3, 15.1±5.5, 32.2±7.9 and 79.3±11.6 in non-irrigated trial while 2.7±2.2, 6.2±2.9, 13.3±5.1, 26.9±7.5 and 76.0±11.2 in irrigated trials. Senescing faster in the non-irrigated (drought stress) trial than the irrigated trial (FIG. 7C). RILs derived from LH82xLAMA senesced fastest as compared to other RIL populations (Ki3xNC356 or Tx740xNC356) (FIG. 7C). There was no dramatic change for DTSE and GFP across trials. DTSE were found to be 130.3±5.0 and 368130.2±6.0, and GFP was found to be 53.5±4.9 and 54.0±5.9 in irrigated and non-irrigated trials were found to be shorter in non-irrigated trial than irrigated trial (FIG. 7D). FIG. 7A illustrates variance component result for temporal senescence progression, FIG. 7B illustrates variance component results for flowering times (DTA, DTS); plant heights (PHT, FHT and EHT), grain filling period (GFP) and days to senescence (DTSE). FIG. 7C illustrates genotypic values by RIL-population for temporal senescence across 85, 91, 100, 111 and 128. FIG. 7D illustrates genotypic values by each RIL population for flowering times (DTA, DTS); plant heights (PHT, FHT and EHT), 378 grain filling period (GFP) and days to senescence (DTSE).

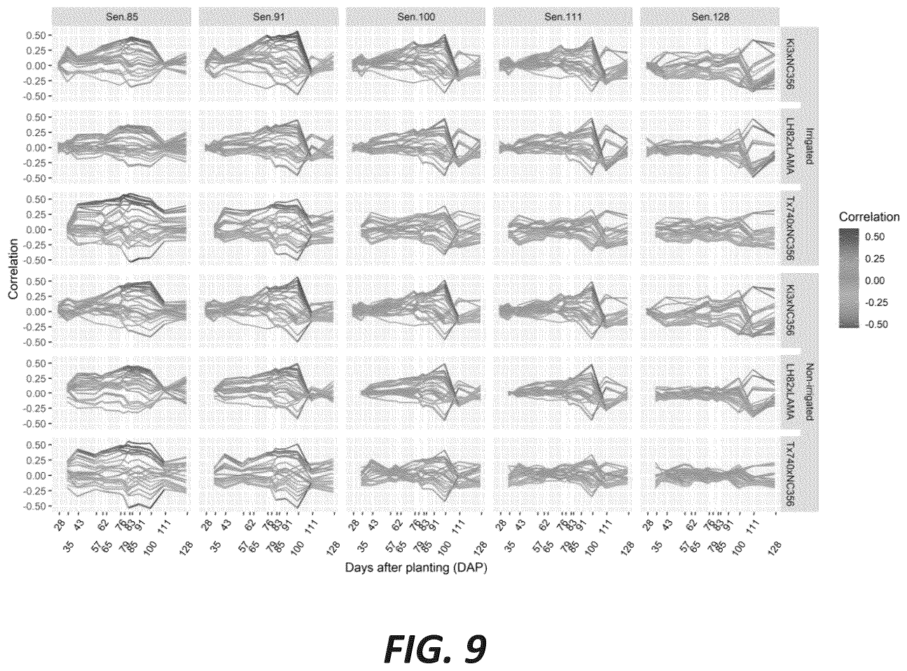

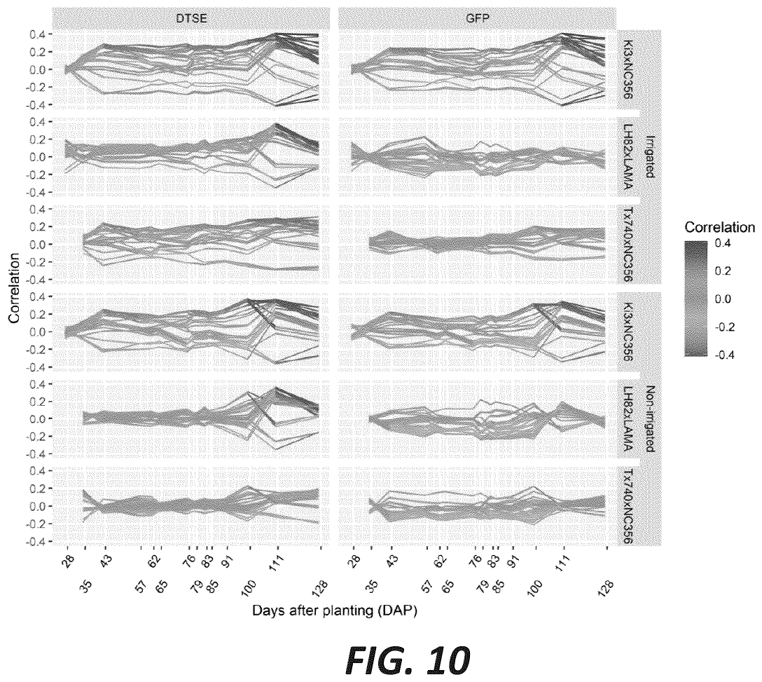

Temporal correlation between temporal senescence and temporal vegetation index temporal values of VIs had dynamic correlations with senescence scores at 85, 91, 100, 111 and 384128 DAPs ranged between −0.5 to 0.5, and differed across bi-parental populations and trials (FIG. 9). Temporal correlations between temporal values of VIs and DTSE and GFP fluctuated between −0.4 and 0.4 (FIG. 10).

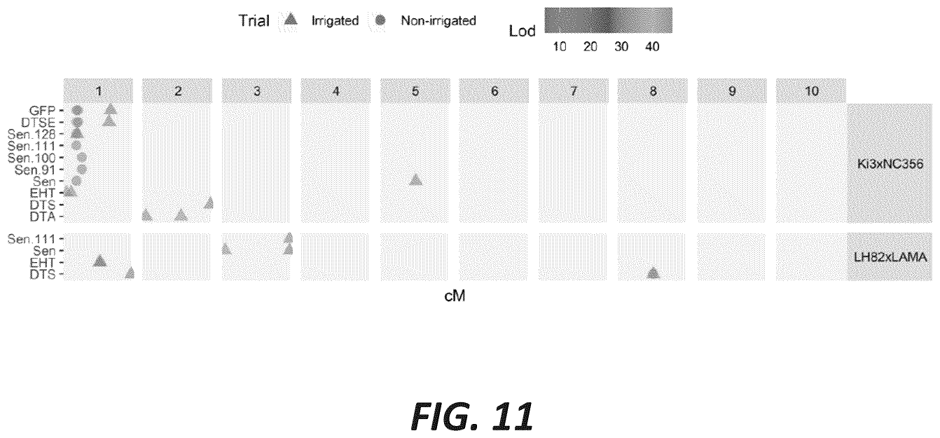

One QTL was discovered for senescence (sen) and senescence at 111 DAPs on chromosome 3 (from 23,845,336 bp to 23,846,812 bp) with the LOD scores of 46 and 28, and 11% and 5% phenotypic variations explained respectively (FIG. 11). One QTL was discovered on chromosome 8 (from 120,610,321 bp to 122,427,978 bp) for DTS in LH82xLAMA population in both trials with LOD scores of ˜6 and ˜8, and ˜12% and 16% phenotypic variations explained respectively (FIG. 11).

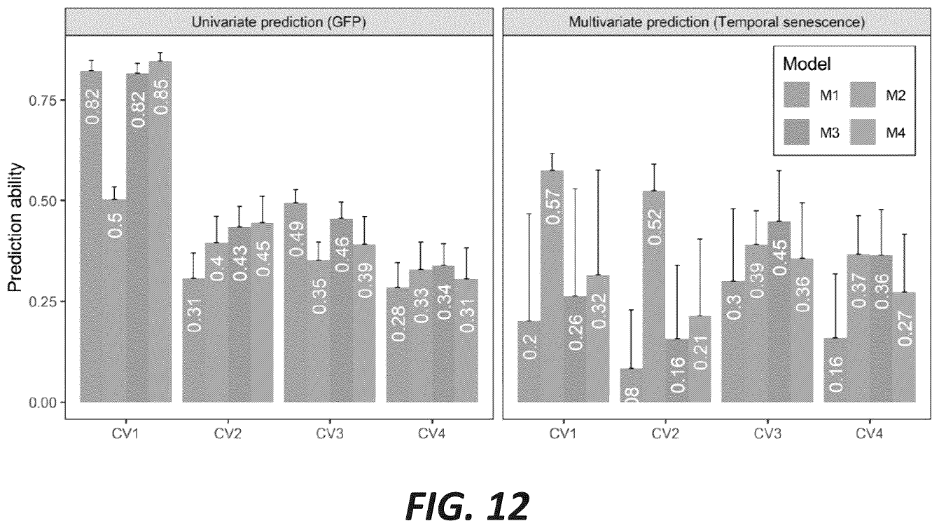

In the univariate prediction of GFP, phenomic biomarker prediction (M2) was more successful than genomic prediction (M1) in predicting untested genotypes in either observed (CV2) or unobserved environments (CV4) (FIG. 12) while genomic prediction was more successful in the CV1 and CV3 scenarios. Combined prediction (M3) best predicted the GFP in untested 428 genotypes in unobserved environments (CV4) (FIG. 12). In the multivariate prediction of temporal senescence, phenomic biomarker prediction (M2) performed best across overall prediction scenarios, except for tested genotypes in untested environments (CV3), in that combined prediction of genomic and phenomic data (M3) performed best (FIG. 12).

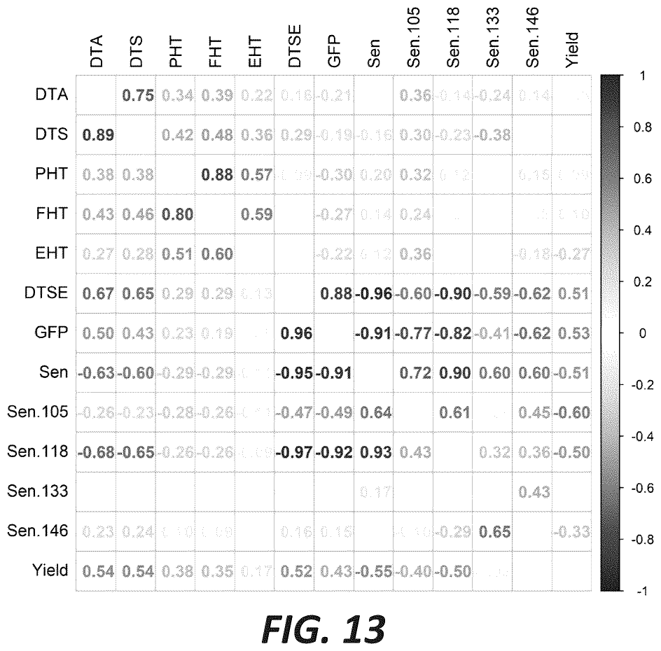

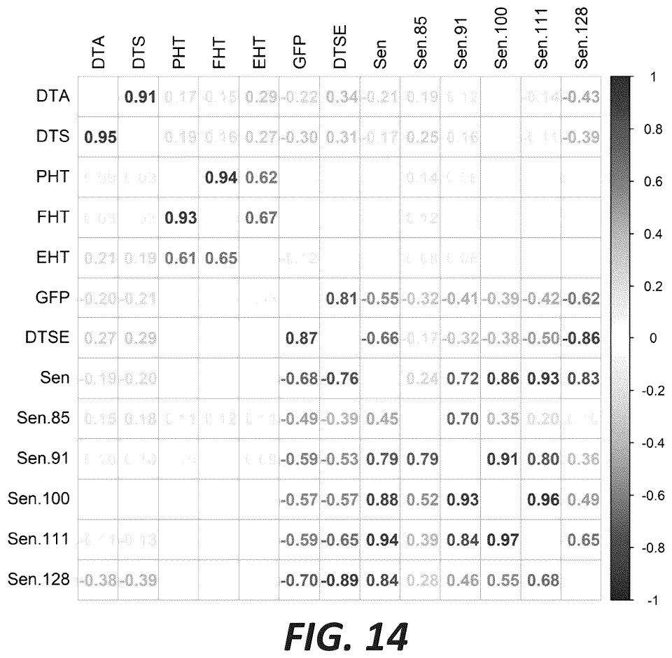

Correlation between temporal senescence, DTSE, GFP and grain yield in RIL and hybrid populations Correlation between GFP and DTSE were found to be stronger in both RIL (0.81 and 0.87 in irrigated and non-irrigated trials) and hybrid trials (0.96) than those between GFP and flowering times (DTA and DTS) (FIG. 13, FIG. 14). Correlations between GFP and temporal senescence were also found to be stronger in the non-irrigated trial than irrigated trial in RIL populations. Correlation between GFP and yield was found to be 0.43 when all hybrids were used; however, correlation increased to 0.53 when only the top ten percent of hybrids were used (FIG. 13). The correlations between yield and DTSE were not affected by either all or top ten percent hybrids and found to be ˜0.52 overall (FIG. 13). The correlation between yield and senescence at day 105 DAPs was the strongest (−0.60) in the top ten percent hybrids. Although a single day (day 105) had a strong correlation and day 118 also had a strong correlation (−0.50), the later days had low to no correlations. Also, no meaningful correlation between yield and flowering was observed, so senescence was the driver of GFP and not flowering (FIG. 13).

Field-based phenotyping facilitates continuous and non-destructive monitoring of plant growth and development throughout the growing season. The longitudinal approach taken here enables the tracking of senescence progression over time, capturing the dynamics and temporal patterns associated with plant aging and responses to environmental conditions. This study demonstrated the modelling of temporal senescence progression using implementation of the present disclosure including a mechanistic growth curve, resulted in discovery of two yield related traits namely days to senescence (DTSE) and grain filling period (GFP) (FIG. 5). These two agronomically novel traits could not be phenotyped previously because insufficiency inefficiency and impracticality using traditional phenotyping (Egli, 2004). However, field-based high throughput phenotyping, combined with temporal modeling paved the way for monitoring and recording of temporal senescence progression (FIG. 7C) and combined with phenomic biomarkers allowed prediction. By monitoring senescence at regular intervals, researchers can model senescence curves and identify specific stages, rates of senescence and the length of the grain filling period for each genotype. The data obtained from field-based high-throughput phenotyping studies can be used to develop predictive models and algorithms that assist plant breeders in selecting genotypes with desirable senescence and grain filling period characteristics. By understanding the relationships between important plant traits and senescence progression, breeders can more effectively develop cultivars with improved senescence patterns, leading to enhanced crop productivity, quality, and resilience.

As a matter of first concern when introducing any new phenotypic measure or trait to plant biology, the heritability of temporal senescence, GFP and DTSE were calculated and found to be more than moderate (FIG. 7A, FIG. 7B). This indicates that these traits can potentially be selected in the prediction model with high selection efficiency. High accuracy in modeling temporal senescence with a mechanistic growth model even in diverse trials (R\hat{ }2=0.98−| and 0.97 in irrigated and non-irrigated RIL/hybrid trials) demonstrated not only the evidence of nonlinearity of temporal senescence progression, but also the wide applicability of the mechanistic growth model of modelling temporal senescence progression in different environmental conditions. Besides identifying the trajectories, modeling the temporal progression helps dilute measurement error at each individual measurement, increasing consistency between experimental replications (Lane and Murray, 2021). A similar temporal modeling approach shown for maize plant height by (Anderson et al., 2020), increased heritability over raw measurements, importantly it also allows measurements in between flight dates to be interpolated.

During the grain filling period, the maize plant undergoes a series of physiological and metabolic changes that result in the accumulation of starch and protein in the developing grain. The plant relies heavily on photosynthesis during this period to produce the energy needed for grain filling (Wang et al., 1999). The stay green feature can play a critical role in sustaining photosynthesis during the grain filling period. When maize plants have a longer stay green period, they can maintain their leaf area and photosynthetic capacity for a longer time, allowing for more photosynthate to be produced and transferred to the developing grain. This, in turn, can result in a longer grain filling period, higher yield, and better grain quality. Therefore, the duration of the grain filling period can influence the expression of the stay green trait in maize plants, and the interaction between these two traits is an essential aspect of maize plant growth and development that could be dissected by the means of FHTP application in this study. Prolonged GFP leads to higher grain yield in grain crops (Egli, 2004), yet effective interplay between source-sink capacity during GFP is crucial to warrant stable grain yield (Abeledo et al., 2020; Albacete et al., 2014). Duration of grain filling period should be taken into consideration along with yield to better understand the crop productivity. Dividing yield by the duration of grain filling period provides insights into the efficiency of yield production over a specific period. It allows for the comparison of different genotypes or management practices in terms of their ability to convert the available time into productive yield. This information is valuable for optimizing crop production systems and identifying high-yielding genotypes that perform well within a specific time frame.

GFP and temporal senescence and staygeen are complex processes that involve multiple physiological and biochemical changes in plants over time. This is why temporal senescence scores had dynamic and useful correlations across growth (FIG. 9), providing time sensitive predictors that genomic data alone could not. Temporal phenomic biomarker data can also provide more accurate predictions of complex traits by capturing the effects of gene-environment interactions, furthermore this can be done in near real time and help with future predictions. Genomic data can provide information about loci associated with a trait, but cannot capture complex interactions between these genes and the environment that influence trait expression cumulatively or across time.

Overall, temporal phenomic data enables a more comprehensive and accurate understanding of complex traits belonging to time periods, such as GFP and temporal senescence progression in plants, than genomic data alone. Combining temporal phenomic biomarker data with genomic data can provide additional insights into the genetic and environmental factors that influence complex trait expression (Anderson et al. 2019).

Univariate and multivariate prediction of GFP and temporal senescence progression in univariate prediction of GFP, phenomic prediction (M2) outperformed the genomic prediction (M1) in the most challenging prediction scenarios in plant breeding programs such as untested genotypes in both observed and unobserved environments (CV2 and CV4 in FIG. 12). In addition, combined prediction performed well in all scenarios (M3 in FIG. 12). In multivariate prediction of temporal senescence progression, phenomic prediction (M2) outperformed genomic prediction (M1) as well as M4 in all prediction scenarios (CV1, CV2, CV3 and CV4).

Overall, the findings of the current study suggest that phenomic biomarker data can capture characteristics of diverse environments better than genomic data alone (FIG. 12). It is essential to emphasize that temporal (longitudinal) phenomic biomarker data collected by UAS refers to the collection of data over time, encompassing various dynamic phenotypic traits of crops and the interactions with the environment. In contrast, genomic data focuses on the genetic information of crops. The underlying reasons why temporal phenomic biomarker data generally predicts complex traits in crops better than genomic data may be attributed to several factors. First is the integration of environmental factors on crop performance. This includes environmentally specific variables such as temperature, humidity, light intensity, and nutrient availability, which can significantly affect crop growth and development. Genomic data alone will not capture the dynamic nature of these interactions, making temporal phenomic biomarker data more informative when environments differ. However, these environmental factors also interact with genetic factors, leading to complex trait variations leading to the second reason of phenotypic plasticity. Crops exhibit phenotypic plasticity, meaning their traits vary based on environmental cues. Temporal phenomic biomarker data allows for the observation and measurement of how crops respond to different environmental conditions over time, but also in near real-time. This information can provide insights into the adaptive capacity of crops, their resilience to stress, and their ability to optimize resource allocation. Genomic data, which represents static genetic information, may not fully capture the potential phenotypic variations and responses to changing conditions. Third is gene-environment interactions. Complex traits in crops are often influenced by interactions between multiple individual genes and environmental factors. These interactions can be dynamic and may vary across different developmental stages. Temporal phenomic biomarker data provides a comprehensive view of how genes and the environment interact over time, enabling the identification of patterns and correlations that contribute to trait expression. Genomic data alone may not reveal the specific gene-environment interactions responsible for complex trait variations. Fourth is the quantitative nature of temporal phenomic biomarker data. Temporal phenomic biomarker data often includes continuous measurements of various traits, allowing for quantitative analysis and the applications of statistical models. This quantitative nature provides a rich dataset for identifying patterns, correlations, and predictive relationships. Genomic data, while providing valuable genetic information, may not possess the same level of quantitative detail necessary for robust prediction of complex traits.

Fifth is non-genetic factors. Complex traits in crops can also be influenced by non-genetic factors such as epigenetic modifications, post-translational modifications, and metabolic processes. These factors are not expected to be fully captured by genomic data but would be expected be fully reflected in temporal phenomic biomarker data. By considering non-genetic factors, temporal phenomic biomarker data provides a more holistic understanding of the underlying mechanisms driving complex trait variation. Overall, the dynamic and quantitative nature of temporal phenomic biomarker data, along with its ability to integrate environmental factors, gene-environment interactions, phenotypic plasticity, and non-genetic factors, contribute to the methods using temporal phenomic biomarker data's superior predictive power for complex traits in crops compared to genomic data alone.

Underlying genetic mechanisms for GFP, DTSE and temporal senescence GFP, DTSE and temporal senescence are critical periods for maize yield and quality, determining the final weight and composition of the grain. The gene GRMZM2G126808, also known as hb45, is a transcription factor in maize and falls into the QTL region with the highest LOD score (LOD=28) in chromosome 1 that was discovered for GFP as well as DTSE and senescence at 128 DAPs in Ki3xNC356 population grown in the non-irrigated trial (FIG. 11). GRMZM2G126808 (hb45) belongs to the homeobox-leucine zipper (HB-Zip) family of transcription factors, which are involved in regulating various aspects of plant growth and development and are expressed at high levels in the embryo and endosperm tissues of developing maize seeds that control kernel length after pollination (Pang et al., 2019; Zhan et al., 2018). Overall, available evidence here suggests for the first time that possibly the reason that the hb45 gene is important for proper kernel development after flowering times in maize is that it influences the duration of grain filling period, and senescence.

The QTL with the second highest LOD score was discovered for senescence (sen; main effect of G in Eq 4.) and senescence at 111 DAPs in LH82xLAMA population on chromosome 3 that harbors only the GRMZM2G378452 gene. GRMZM2G378452 encodes the protein kinase domain-containing protein (PKDs); PKDs have previously been shown to play a role in regulating senescence and timing of reproductive stage in soybean (Xu et al., 2011). One other locus of note was around the chromosome containing the ZCN8 gene, discovered for DTS in LH82xLAMA population growing in both irrigated and non-irrigated trials. The ZCN8 gene is known as a maize florigen gene (Meng et al., 2011). It is interesting that ZCN8 was only discovered for flowering time but neither temporal senescence, GFP nor DTSE. In addition, no other consistent QTL were discovered for flowering time that also effected temporal senescence, GFP or DTSE. Yet a few QTLs were discovered for combinations between GFP, DTSE and temporal senescence (FIG. 11). This shows that GFP and temporal senescence can be governed by the same major QTLs (and likely minor loci not detected in this study), indicating that genetic modification for temporal senescence can directly influence the GFP in this germplasm. Yet no such relationship between GFP and flowering time was detected, lacking common QTL between flowering times and GFP. GFP and temporal senescence progression are directly linked since the GFP occurs during senescence progression, when the plant remobilized nutrients to the developing grain. Thus, any factors that affect the progression of senescence can impact the GFP and ultimately the yield and quality of the grain. In contrast, flowering time is more closely linked to the reproductive development of the plant, and its relationship to the GFP is more indirect. The timing of flowering can affect the duration of the GFP, but this relationship can be influenced by external factors such as diverse environmental conditions less so than genetic factors.

The study included physiological evaluation of grain filling period. Correlations between GFP and DTSE (0.81 and 0.87 in irrigated and non-irrigated trials) were substantially greater than correlations between GFP and days to flowering (−0.22, −0.20 for DTA, −0.30, −0.21 for DTS in irrigated and non-irrigated trials) (FIG. 14). This combined with QTL results suggests that GFP is physiological process primarily driven by senescence or stay green in the Texas environment, less affected by genomic variation of days to flowering. Senescence and GFP have both been shown in diverse hybrid trials to be closely linked to the final grain weight (FIG. 13, (DeSalvio et al., 2022; Wang et al., 1999)). During senescence, the plant mobilizes nutrients and energy from leaves and other vegetative tissues to the developing grain, the main sinks for these resources. The duration of the grain filling period in maize is determined by physiological processes occurring after pollination. Therefore, based on the results of these genetics and environments, even if the plant flowers early or late, the duration of the grain filling period may not be significantly affected. In summary, while days to flowering can indirectly impact the grain filling period through its effects on pollination and fertilization, it may not have a direct influence on the duration of the grain filling period.

### REFERENCES

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.
Abeledo L G, Savin R, Slafer G A. 2020. Maize senescence under contrasting source-sink ratios during the grain filling period. Environmental and Experimental Botany 180, 104263.
Adak A, Anderson S L, Murray S C. 2023. Pedigree-management-flight interaction for temporal phenotype analysis and temporal phenomic prediction. The Plant Phenome Journal 6, e20057.
Adak A, Murray S C, Anderson S L. 2022. Temporal phenomic predictions from unoccupied aerial systems can outperform genomic predictions. G3 Genes|Genomes/Genetics 13. Adak A, Murray S C, Božinović S, Lindsey R, Nakasagga S, Chatterjee S, Anderson S L, Wilde S. 2021. Temporal Vegetation Indices and Plant Height from Remotely Sensed Imagery Can Predict Grain Yield and Flowering Time Breeding Value in Maize via Machine Learning Regression. Remote Sensing 13, 2141.
Albacete A A, Martínez-Andújar C, Pérez-Alfocea F. 2014. Hormonal and metabolic regulation of source-sink relations under salinity and drought: From plant survival to crop yield stability. Biotechnology Advances 32, 12-30. Extraction of Research Plot Scale Agriculture Remote Sensing Data. Frontiers in Plant Science 11. aerial system enabled functional modeling of maize height reveals dynamic expression of loci. Plant direct 4, e00223.
Araus J L, Cairns J E. 2014. Field high-throughput phenotyping: the new crop breeding frontier. Trends in plant science 19, 52-61.
Araus J L, Kefauver S C, Zaman-Allah M, Olsen M S, Cairns J E. 2018. Translating high-throughput phenotyping into genetic gain. Trends in plant science 23, 451-466. Mueller-Roeber B. 2010. A gene regulatory network controlled by the NAC transcription factor ANAC092/AtNAC2/ORE1 during salt-promoted senescence. Plant J 62, 250-264. Capristo P R, Rizzalli R H, Andrade F H. 2007. Ecophysiological Yield Components of Maize Hybrids with Contrasting Maturity. Agronomy Journal 99, 1111-1118. alleles that delay senescence to extend wheat grain fill duration. J Exp Bot 72, 7710-7728. applications. Plant Molecular Biology Reporter 17, 53-57. Mays L.) and validating SNPs controlling maize grain yield and plant height in southern hybrid testcrosses.
Ciampitti I A, Vyn T J. 2013. Grain Nitrogen Source Changes over Time in Maize: A Review. Crop science 692 53, 366-377. Crain J, Mondal S, Rutkoski J, Singh R P, Poland J. 2018. Combining high-throughput phenotyping and genomic information to increase prediction and selection accuracy in wheat breeding. Plant genome 11, 1-14.
DeSalvio A J, Adak A, Murray S C, Wilde S C, Isakeit T. 2022. Phenomic data-facilitated rust and senescence prediction in maize using machine learning algorithms. Scientific Reports 12, 7571. Egli D B. 2004. Seed-fill duration and yield of grain crops. In: Sparks D L, ed. Advances in agronomy, Vol. 83: Elsevier, 243-279.
Farfan I D B, De La Fuente G N, Murray S C, Isakeit T, Huang P-C, Warburton M, Williams P, Windham G L, Kolomiets M. 2015. Genome wide association study for drought, aflatoxin resistance, and important agronomic traits of maize hybrids in the sub-tropics. PloS one 10, e0117737.
Fu J D, Yan Y F, Kim M Y, Lee S H, Lee B W. 2011. Population-specific quantitative trait loci mapping for functional stay-green trait in rice (Oryza sativa L.). Genome 54, 235-243.
Fukao T, Yeung E, Bailey-Serres J. 2012. The Submergence Tolerance Gene SUB1A Delays Leaf Senescence under Prolonged Darkness through Hormonal Regulation in Rice Plant Physiology 160, 1795-1807.
Galán R J, Bernal-Vasquez A-M, Jebsen C, Piepho H-P, Thorwarth P, Steffan P, Gordillo A, Miedaner T. 2020. Integration of genotypic, hyperspectral, and phenotypic data to improve biomass yield prediction in hybrid rye. Theoretical and Applied Genetics 133, 3001-3015.
Galán R J, Bernal-Vasquez A-M, Jebsen C, Piepho H-P, Thorwarth P, Steffan P, Gordillo A, Miedaner T. 2021. Early prediction of biomass in hybrid rye based on hyperspectral data surpasses genomic predictability in less-related breeding material. Theoretical and Applied Genetics 134, 1409-1422. Gepstein S, Sabehi G, Carp M J, Hajouj T, Nesher M F, Yariv I, Dor C, Bassani M. 2003. Large-scale identification of leaf senescence-associated genes. Plant J 36, 629-642.
Ghanem M E, Albacete A, Martínez-Andújar C, Acosta M, Romero-Aranda R, Dodd I C, Lutts S, PérezAlfocea F. 2008. Hormonal changes during salinity-induced leaf senescence in tomato (Solanum lycopersicum L.). Journal of Experimental Botany 59, 3039-3050.
Gonda I, Ashrafi H, Lyon D A, Strickler S R, Hulse-Kemp A M, Ma Q, Sun H, Stoffel K, Powell A F, Futrell S, Thannhauser T W, Fei Z, Van Deynze A E, Mueller L A, Giovannoni J J, Foolad M R. 2019. Sequencing Based Bin Map Construction of a Tomato Mapping Population, Facilitating High-Resolution Quantitative Trait Loci Detection. The Plant Genome 12, 180010.
Gregersen P L, Culetic A, Boschian L, Krupinska K. 2013. Plant senescence and crop productivity. Plant Mol Biol 82, 603-622.
Gregersen P L, Holm P B. 2007. Transcriptome analysis of senescence in the flag leaf of wheat (Triticum aestivum L.). Plant Biotechnol J 5, 192-206.
Großkinsky D K, Syaifullah S J, Roitsch T. 2017. Integration of multi-omics techniques and physiological phenotyping within a holistic phenomics approach to study senescence in model and crop plants. Journal of Experimental Botany 69, 825-844.
Hassan M A, Yang M, Rasheed A, Tian X, Reynolds M, Xia X, Xiao Y, He Z. 2021. Quantifying senescence in bread wheat using multispectral imaging from an unmanned aerial vehicle and QTL mapping. Plant Physiology 187, 2623-2636.
Herr A W, Adak A, Carroll M E, Elango D, Kar S, Li C, Jones S E, Carter A H, Murray S C, Paterson A, Sankaran S, Singh A, Singh A K. Unoccupied aerial systems imagery for phenotyping in cotton, maize, soybean, and wheat breeding. Crop science n/a. Jacquemoud S, Baret F. 1990. PROSPECT: A model of leaf optical properties spectra. Remote sensing of Environment 34, 75-91.
Jarquin D, Crossa J, Lacaze X, Du Cheyron P, Daucourt J, Lorgeou J, Piraux F, Guerreiro L, Pérez P, Calus M, Burgueño J, de los Campos G. 2014. A reaction norm model for genomic selection using high dimensional genomic and environmental data. Theoretical and Applied Genetics 127, 595-607. Juliana P, Montesinos-López O A, Crossa J, Mondal S, González Pérez L, Poland J, Huerta-Espino J, Crespo-Herrera L, Govindan V, Dreisigacker S, Shrestha S, Pérez-Rodríguez P, Pinto Espinosa F, Singh R P. 2019. Integrating genomic-enabled prediction and high-throughput phenotyping in breeding for climate-resilient bread wheat. Theoretical and Applied Genetics 132, 177-194. Krause M R, González-Pérez L, Crossa J, Pérez-Rodríguez P, Montesinos-López O, Singh R P, Dreisigacker S, Poland J, Rutkoski J, Sorrells M, Gore M A, Mondal S. 2019. Hyperspectral Reflectance-Derived Relationship Matrices for Genomic Prediction of Grain Yield in Wheat. G3 Genes/Genomes|Genetics 9, 1231-1247.
Krause M R, Mondal S, Crossa J, Singh R P, Pinto F, Haghighattalab A, Shrestha S, Rutkoski J, Gore M A, Sorrells M E, Poland J. 2020. Aerial high-throughput phenotyping enables indirect selection for grain yield at the early generation, seed-limited stages in breeding programs. Crop science 60, 3096-3114. Lane H M, Murray S C. 2021. High throughput can produce better decisions than high accuracy when phenotyping plant populations. Crop science 61, 3301-3313.
Lauter N, Kampani A, Carlson S, Goebel M, Moose S P. 2005. microRNA172 down-regulates <i > glossy 15</i > to promote vegetative phase change in maize. Proceedings of the National Academy of Sciences 102, 9412-9417.
Lee S, Seo P J, Lee H J, Park C M. 2012. A NAC transcription factor NTL4 promotes reactive oxygen species production during drought-induced leaf senescence in Arabidopsis. Plant J 70, 831-844.
Li H, Ye G, Wang J. 2007. A Modified Algorithm for the Improvement of Composite Interval Mapping. Genetics 175, 361-374.
Lim P O, Woo H R, Nam H G. 2003. Molecular genetics of leaf senescence in Arabidopsis. Trends in plant science 8, 272-278.
Liu L, Zhou Y, Zhou G, Ye R, Zhao L, Li X, Lin Y. 2008. Identification of early senescence-associated genes in rice flag leaves. Plant Mol Biol 67, 37-55.
Makanza R, Zaman-Allah M, Cairns J E, Magorokosho C, Tarekegne A, Olsen M, Prasanna B M. 2018. High-Throughput Phenotyping of Canopy Cover and Senescence in Maize Field Trials Using Aerial Digital Canopy Imaging. Remote Sensing 10, 330.
Matias F I, Caraza-Harter M V, Endelman J B. 2020. FIELDimageR: an R package to analyze orthomosaic images from agricultural field trials. The Plant Phenome Journal 3, e20005.
Meng L, Li H, Zhang L, Wang J. 2015. QTL IciMapping: Integrated software for genetic linkage map construction and quantitative trait locus mapping in biparental populations. The Crop Journal 3, 269283.
Meng X, Muszynski M G, Danilevskaya O N. 2011. The F T-like ZCN8 gene functions as a floral activator and is involved in photoperiod sensitivity in maize. The Plant Cell 23, 942-960.
Messmer R, Fracheboud Y, Banziger M, Stamp P, Ribaut J-M. 2011. Drought stress and tropical maize: QTLs for leaf greenness, plant senescence, and root capacitance. Field Crops Research 124, 93-103. Montesinos-López A, Montesinos-López O A, Cuevas J, Mata-López W A, Burgueño J, Mondal S, Huerta J, Singh R, Autrique E, González-Pérez L, Crossa J. 2017. Genomic Bayesian functional regression models with interactions for predicting wheat grain yield using hyper-spectral image data. Plant Methods 13, 62. Pang J, Fu J, Zong N, Wang J, Song D, Zhang X, He C, Fang T, Zhang H, Fan Y, Wang G, Zhao J. 2019. Kernel size-related genes revealed by an integrated eQTL analysis during early maize kernel development. The Plant Journal 98, 19-32.
Pérez-Alfocea F, Albacete A, Ghanem M E, Dodd I C. 2010. Hormonal regulation of source sink relations to maintain crop productivity under salinity: a case study of root-to-shoot signalling in tomato. Functional Plant Biology 37, 592-603.
Pérez-Rodríguez P, de los Campos G. 2022. Multitrait Bayesian shrinkage and variable selection models with the BGLR-R package. Genetics 222. Pérez P, de los Campos G. 2014. Genome-Wide Regression and Prediction with the BGLR Statistical Package. Genetics 198, 483-495.
Pommel B, Gallais A, Coque M, Quillere I, Hirel B, Prioul J L, Andrieu B, Floriot M. 2006. Carbon and nitrogen allocation and grain filling in three maize hybrids differing in leaf senescence. European Journal of Agronomy 24, 203-211.
Raineri J, Wang S, Peleg Z, Blumwald E, Chan R L. 2015. The rice transcription factor OsWRKY47 is a positive regulator of the response to water deficit stress. Plant Mol Biol 88, 401-413.
Rivero R M, Shulaev V, Blumwald E. 2009. Cytokinin-Dependent Photorespiration and the Protection of Photosynthesis during Water Deficit Plant Physiology 150, 1530-1540.
Rutkoski J, Poland J, Mondal S, Autrique E, Pérez L G, Crossa J, Reynolds M, Singh R. 2016. Canopy temperature and vegetation indices from high-throughput phenotyping improve accuracy of pedigree and genomic selection for grain yield in wheat. G3: Genes, Genomes, Genetics 6, 2799-2808. Sade N, del Mar Rubio-Wilhelmi M, Umnajkitikorn K, Blumwald E. 2017. Stress-induced senescence and plant tolerance to abiotic stress. Journal of Experimental Botany 69, 845-853.
Sade N, Del Mar Rubio-Wilhelmi M, Umnajkitikorn K, Blumwald E. 2018. Stress-induced senescence and plant tolerance to abiotic stress. J Exp Bot 69, 845-853.
Sakuraba Y, Park S Y, Kim Y S, Wang S H, Yoo S C, Hörtensteiner S, Paek N C. 2014. Arabidopsis STAYGREEN2 is a negative regulator of chlorophyll degradation during leaf senescence. Mol Plant 7, 12881302.
Schippers J H M, Jing H-C, Hille J, Dijkwel P P. 2007. Developmental and hormonal control of leaf senescence. In: Gan S, ed. Senescence Processes in Plants: Blackwell Publishing, 145-170.
Schippers J H M, Schmidt R, Wagstaff C, Jing H-C. 2015. Living to Die and Dying to Live: The Survival Strategy behind Leaf Senescence. Plant Physiology 169, 914-930.
Schnable P S, Ware D, Fulton R S, Stein J C, Wei F, Pasternak S, Liang C, Zhang J, Fulton L, Graves T A. 2009. The B73 maize genome: complexity, diversity, and dynamics. science 326, 1112-1115.
Shi Y, Thomasson J A, Murray S C, Pugh N A, Rooney W L, Shafian S, Rajan N, Rouze G, Morgan C L, Neely H L. 2016. Unmanned aerial vehicles for high-throughput phenotyping and agronomic research. PloS one 11, e0159781.
Sun J, Poland J A, Mondal S, Crossa J, Juliana P, Singh R P, Rutkoski J E, Jannink J-L, Crespo-Herrera L, Velu G, Huerta-Espino J, Sorrells M E. 2019. High-throughput phenotyping platforms enhance genomic selection for wheat grain yield across populations and cycles in early stage. Theoretical and Applied Genetics 132, 1705-1720.
Sun J, Rutkoski J E, Poland J A, Crossa J, Jannink J-L, Sorrells M E. 2017. Multitrait, Random Regression, or Simple Repeatability Model in High-Throughput Phenotyping Data Improve Genomic Prediction for Wheat Grain Yield. The Plant Genome 10, plantgenome2016.2011.0111.
Tsanakas G F, Manioudaki M E, Economou A S, Kalaitzis P. 2014. De novo transcriptome analysis of petal senescence in Gardenia jasminoides Ellis. BMC Genomics 15, 554.
VanRaden P M. 2008. Efficient Methods to Compute Genomic Predictions. Journal of Dairy Science 91, 4414-4423.
Wang G, Kang M S, Moreno O. 1999. Genetic analyses of grain-filling rate and duration in maize1Approved for publication by the director of the Louisiana Agricultural Experiment Station as manuscript no. 98-09-0288.1. Field Crops Research 61, 211-222.
Woo H R, Kim H J, Nam H G, Lim P O. 2013. Plant leaf senescence and death-regulation by multiple layers of control and implications for aging in general. Journal of Cell Science 126, 4823-4833. Wu X, Ding D, Shi C, Xue Y, Zhang Z, Tang G, Tang J. 2016. microRNA-dependent gene regulatory networks in maize leaf senescence. BMC Plant Biology 16, 73. Xu F, Meng T, Li P, Yu Y, Cui Y, Wang Y, Gong Q, Wang N N. 2011. A Soybean Dual-Specificity Kinase, GmSARK, and Its Arabidopsis Homolog, AtSARK, Regulate Leaf Senescence through Synergistic Actions of Auxin and Ethylene Plant Physiology 157, 2131-2153.
Yang J C, Zhang J H, Wang Z Q, Zhu Q S, Liu L. 2003. Involvement of abscisic acid and cytokinins in the senescence and remobilization of carbon reserves in wheat subjected to water stress during grain filling. Plant, Cell & Environment 26, 1621-1631.
Yolcu S, Li X, Li S, Kim Y J. 2017. Beyond the genetic code in leaf senescence. Journal of Experimental 841 Botany 69, 801-810.
Young T E, Meeley R B, Gallie D R. 2004. ACC synthase expression regulates leaf performance and drought tolerance in maize. The Plant Journal 40, 813-825.
Zhan J, Li G, Ryu C-H, Ma C, Zhang S, Lloyd A, Hunter B G, Larkins B A, Drews G N, Wang X, Yadegari R. 2018. Opaque-2 Regulates a Complex Gene Network Associated with Cell Differentiation and Storage Functions of Maize Endosperm. The Plant Cell 30, 2425-2446.
Zheng Y, Wang Z, Sun X, Jia A, Jiang G, Li Z. 2008. Higher salinity tolerance cultivars of winter wheat relieved senescence at reproductive stage. Environmental and Experimental Botany 62, 129-138.

1. A method of phenotypic measurement comprising:
receiving image data from a remote sensing platform, wherein the image data comprises a plurality of temporally-spaced images of a plant;
inputting the image data into a prediction model; and
determining, using the prediction model, a phenotypic measurement.

2. The method of claim 1, wherein the prediction model comprises a phenomic biomarker prediction model.

3. The method of claim 1, wherein the prediction model comprises a genomic, metabolomic, or proteomic prediction model.

4. The method of claim 1, wherein the prediction model is a trained machine learning model.

5. The method of claim 1, wherein the phenotypic measurement of interest comprises plant senescence or stay green.

6. The method of claim 1, further comprising determining a senescence score based on the plurality of image data.

7. The method of claim 1, further comprising determining a dependent phenotypic trait of interest, wherein the dependent phenotypic trait of interest comprises grain filling period.

8. The method of claim 1, further comprising modeling a trajectory of the phenotypic measurement using a mathematical or statistical model over time.

9. The method of claim 1, further comprising controlling the remote sensing platform based on the phenotypic measurement.

10. The method of claim 1, further comprising adjusting the inputs to the plant based on the phenotypic measurement.

11. The method of claim 1, further comprising selecting varieties based on phenomic prediction of senescence and stay green.

12. A system comprising:
a remote sensing platform comprising a remote sensor configured to capture image data, wherein the image data comprises a plurality of temporally-spaced images of a plant; and
a computing device operably coupled to the remote sensing platform, wherein the computing device comprises at least one processor and memory, the memory having computer-executable instructions stored thereon that, when executed by the at least one processor, cause the at least one processor to:
input the image data into a prediction model; and
determine, using the prediction model, a phenotypic measurement.

13. The system of claim 12, wherein the prediction model comprises a phenomic biomarker prediction model.

14. The system of claim 12, wherein the prediction model comprises a genomic, metabolomic, or proteomic prediction model.

15. The system of claim 12, wherein the prediction model is a trained machine learning model.

16. The system of claim 12, wherein the phenotypic measurement comprises plant senescence.

17. The system of claim 12, further comprising determining a dependent phenotypic trait of interest, wherein the dependent phenotypic trait of interest comprises grain filling period.

18. The system of claim 12, wherein the memory has further computer-executable instructions stored thereon that, when executed by the at least one processor, cause the at least one processor to: control the remote sensing platform based on the phenotypic measurement.

19. The system of claim 12, wherein the memory has further computer-executable instructions stored thereon that, when executed by the at least one processor, cause the at least one processor to: adjust the inputs to the plant based on the phenotypic measurement.

20. The system of claim 12, wherein the memory has further computer-executable instructions stored thereon that, when executed by the at least one processor, cause the at least one processor to: determine a senescence score or grain filling period based on the plurality of image data.
